(19) United States
(12) Patent Application Publication
(10) Pub. No.: US 2026/0201460 A1
(43) Pub. Date: Jul. 16, 2026

(54) METHODS AND COMPOSITIONS FOR MITIGATING INDEX HOPPING IN DNA SEQUENCING

(71) Applicant: Chapter Diagnostics, Inc., Menlo Park, CA (US)

(72) Inventors: Chunlin Wang, Menlo Park, CA (US); Zhihai Ma, Mountain View, CA (US); Baback Gharizadeh, Menlo Park, CA (US)

(73) Assignee: Chapter Diagnostics, Inc., Menlo Park, CA (US)

(21) Appl. No.: 19/135,030

(22) PCT Filed: Dec. 4, 2023
(86) PCT No.: PCT/US2023/000038

§ 371 Date: Jun. 2, 2025

Related U.S. Application Data
(60) Provisional application No. 63/385,860, filed on Dec. 2, 2022.

(51) Int. Cl. C12Q 1/6869; C12Q 1/6806; C12Q 1/686; C12Q 1/6876

(52) U.S. Cl. CPC C12Q 1/6869; C12Q 1/6806; C12Q 1/686; C12Q 1/6876

(57) ABSTRACT

The present disclosure relates to a method of quadruple combinatorial indexing to mitigate the risk of index hopping when a large number of samples are analyzed in the same next-generation sequencing run. This disclosure describes a method to add unique sequences, called indexes, to each DNA fragment during library preparation, to allow a large number of samples to be analyzed in the same sequencing run with minimal cost and to minimize the risk of assignment of sequencing reads to the wrong sample during demultiplexing. The present disclosure provides methods, compositions, kits, systems, algorithm, and instruments that will mitigate the risk of index hopping when analyzing a large number of nucleic acid samples in the same next-generation sequencing run in comparison to conventional methodologies.

### RELATED APPLICATIONS

This application claims priority to U.S. Provisional Patent Application No. 63/385,860 filed on Dec. 2, 2022, titled “Dual Combination of Unique Indexing Method”, the entire content of which is incorporated herein.

### BACKGROUND OF THE INVENTION

Targeted DNA sequencing allows the user to selectively analyze specific regions of a genome. Instead of sequencing the entire genome, only specific regions of interest are sequenced. This approach is more focused and efficient, as it allows people skilled in the art to gather information about specific genes or genomic regions without sequencing the entire genome. Targeted DNA sequencing typically involves the use of capture probes or primers that are designed to specifically bind to and capture the regions of interest. These primers or probes are often complementary to the DNA sequences being targeted, enabling their selective amplification and sequencing. This is particularly useful when studying specific genes or genomic regions that are known to be associated with certain infections, pathogenicity, diseases, or traits. By targeting these regions, researchers can analyze variations, mutations, or structural changes that may be relevant to a particular condition or characteristic. Compared to whole genome sequencing (WGS), targeted DNA sequencing is faster, less expensive, and requires fewer computational resources. It has become a valuable tool in various research areas, including cancer genomics, genetic disease diagnosis, forensics, and personalized medicine.

The multiplex PCR (polymerase chain reaction) method allows for the simultaneous amplification of multiple target DNA sequences in a single reaction. It involves the use of multiple primer pairs, each specific to a different target sequence, along with a DNA polymerase enzyme and nucleotides. By incorporating multiple primer sets, each corresponding to a specific target region, into a single PCR reaction, multiple DNA fragments can be amplified simultaneously.

Multiplex PCR has broad applications in various fields, including medical diagnostics and sciences, genetics, forensics, microbial and viral detection, food safety testing and food pathogens, drug resistance, pharmacogenetics, environmental testing, epigenetics, allergen testing, botany, ecology, evolutionary biology, genetics, zoology, research, etc.

By simultaneously amplifying multiple target genes or regions, multiplex PCR enables the detection of multiple disease-associated variants in a single test. For pathogen identification, multiplex PCR can be employed for rapid detection and identification of pathogens, such as bacteria, viruses, parasites, or fungi, in clinical, food or environmental samples. By targeting specific genomic regions unique to different pathogens, multiplex PCR can provide a rapid and accurate diagnosis. For cancer mutation analysis, multiplex PCR can be employed to detect specific mutations or genetic alterations associated with cancer. By amplifying target genes known to harbor cancer-associated mutations, multiplex PCR allows for efficient screening and profiling of tumor samples.

Next-generation sequencing (NGS) technology can generate millions to billions of sequencing reads. A key to utilizing this increased capacity is addition of unique nucleic acid sequences, called indexes or barcodes, to each DNA fragment during library preparation during multiplex PCR, as disclosed herein. This allows large numbers of libraries to be pooled and sequenced simultaneously during a single sequencing run. However, gains in throughput from multiplexing come with an added layer of complexity, as sequencing reads from pooled libraries need to be identified and sorted computationally in a process called demultiplexing before final data analysis.

DNA sequencing technologies have significantly advanced our understanding of genetics and genomics. However, one of the challenges in high-throughput sequencing processes is “index hopping” (also called “index switching”, “index swapping”, “barcode jumping”, “index misassignment”, or “sample bleeding”), where sequences from one sample are erroneously assigned to another sample. This can lead to data contamination and inaccurate results in genomics research. The present disclosure aims to mitigate index hopping and improve the reliability of DNA sequencing. Although the overall rate of index hopping is relatively low, it has been seen at elevated levels on certain instruments that use patterned flow cells with exclusion amplification chemistry versus those that do not use patterned flow cells. Even a relatively low rate of index hopping can affect many diagnostic applications using NGS systems, especially for detection, screening of microbial and viral agents or investigating low-copy-number variants and cfDNA and ctDNA. Therefore, a method to eliminate or mitigate this risk is needed.

The typical levels of index hopping for certain sequencing platforms range from 0.1-2% depending on the type, quality, and handling of the library, however, index hopping rates of up to 10% have been reported. Use of unique dual indexing combinations, where target sequences were labeled with unique index sequences at both 5′-end and 3′-end, can substantially decrease the levels of index hopping. However, the operational complexity of this unique dual indexing approach might substantially increase the chance of cross-contamination.

Sequencing biological samples at scale requires a highly streamlined workflow that makes the most efficient use of instrument output and eliminates effects of lane-to-lane variability. To achieve the maximum cost efficiency, sample multiplexing on a sequencer has become a necessity. There are many reports, however, that certain amplification chemistry can lead to serious data integrity issues due to the phenomenon of index hopping. Some studies have reported that the index hopping is likely due to residual excess free primer or adapters in the samples that can lead to spurious extension of library fragments with an oligo containing the wrong sample index. As this phenomenon is a property of the flow cell chemistry itself, the only option to completely eliminate the effects of index hopping is to sequence one sample per lane, which unfortunately is not financially feasible in many cases.

As noted above, although NGS-based targeted amplicon sequencing is a powerful approach, different errors and biases such as variation in sequencing depth between individual samples, sequencing errors rates and index hopping can play an important role within the analysis of NGS data. There are currently no standards requiring detailed reports and explanations to correct such potential errors. Furthermore, use of NGS platforms is increasing by sequencing companies, core facilities, diagnostic laboratories, research institutes and other entities. NGS services provided by third parties often only provide the sequencing data while excluding general information on the NGS run, demultiplexing efficiency of individual samples and other relevant parameters.

Presently, a widely used approach to study large sample numbers is the analysis of pooled samples, by combining DNA from multiple individuals into one sample of the NGS library, thereby excluding the opportunity of backtracking specific sequences to an individual sample. As an example, in microbial and viral detection and screening, the analysis of such pooled samples may lead to inaccurate results due to index hopping. The present disclosure, in contrast, is a cost-efficient NGS protocol and is scalable to hundreds to thousands of individual samples, making it ideal for any study that deals with high sample numbers.

With index hopping, index misassignment between multiplexed libraries and its rate rises as more free adapters or primers are present in the prepared NGS library. To combat this issue, some methods differentiate between combinatorial dual indexing and unique dual indexing. Special kits can be used with unique dual index sequences (e.g., set of 96 primer pairs) to counter the problem of index hopping and the pitfalls of demultiplexing. This is an option for low sample numbers but if several hundred samples are to be individually indexed in one sequencing run, it can be difficult to implement unique dual indexing due to the high number of samples and for cost reasons. For instance, in silico cross-contamination between samples from different studies and altered or falsified results can occur if a flow cell lane is shared and the reads were incorrectly assigned. Even where samples are run exclusively on a single flow cell, index hopping may result in barcode switching events between samples that lead to misassignment of reads.

For certain library preparations of NGS runs, two indexes are often used to tag the individual samples (dual indexing). The sequences are then demultiplexed and the sequenced data are converted into FASTQ file formats. People skilled in the art performing the bioinformatic analysis with demultiplexed data assume that the assignment of the sequences to samples are correct. Verifying this is extremely difficult, because the provided data sets lack all the information on the demultiplexing settings, and moreover, on the extent of sequencing errors within indexes and index hopping. Consequently, sequences can be incorrectly assigned to samples and, in case of a shared flow cell, even across sample sets. However, it is known that demultiplexing errors occur and depend on various factors such as the sequencing platform, the library type used and index combinations. The few existing studies investigating index hopping in more detail give rates of 0.2-10%. The problem of sequencing errors within indexes and index hopping can become particularly significant when many samples with different indexes are sequenced together, In general, when it comes to analyzing sequence data, there are no standard approaches or know-how to handle index hopping in regards to the sequencing platform model used as well as the software tools, which can significantly impact the results and thereby the interpretation. of the sequencing information especially in diagnostic, detection and screening samples. Given this lack of solution, it is a challenge to understand what has been done during sample processing and data analysis, and impossible to compare the outcomes of different studies. To date, published NGS studies, such as targeted amplicon sequencing are difficult to compare or evaluate because of the lack of this essential information on data processing. Without an efficient tool to remove index hopping, there is the possibility of incorrectly assigning samples. Despite implementing sound lab practices to reduce residual adapter or primer, even methods with less than 1% index swapping could still generate spurious results in downstream data analysis.

Index hopping leads to improper demultiplexing, with reads being assigned to the wrong samples which manifests as downstream read contamination in the data. Elimination of index hopping is of paramount importance for any sequencing studies. While a lower rate of index hopping may not affect the ability to trust variant calling for many germline DNA applications, it can lead to spurious results when looking for rare transcripts, fusion events in RNA-seq or low allele fraction somatic analysis or detection, or screening and detection of microbial and viral pathogens. While mutation callers such as data analysis software tools filter for many common artifacts that occur during the sequencing process, index swapped reads are unique as they are high quality reads, not errors, that are assigned to the wrong sample. In cancer genomics studies, there are applications such as blood biopsy using circulating tumor DNA (ctDNA), where researchers try to detect mutations in ctDNA at allele fractions of 5% or lower against a background of normal DNA. If this level of high sensitivity and confidence is required, an elimination or mitigation approach is a necessity as even low rates of sample cross-contamination would hinder the accuracy and sensitivity of low allele fraction variant calling. It should be noted that the phenomenon of index hopping can occur in any scenario where multiplexed libraries are amplified together in the same sequencer and residual adapters and active polymerases are present. When designing sequencing experiments, it is therefore important to keep in mind that any time samples are amplified together in a pool, whether in a tube during library prep or on a flow cell, there is a danger of index hopping induced cross-contamination.

As an example, Costello et al (doi: 10.1186/s12864-018-4703-0) demonstrated that index hopping lead to incorrect assignment of reads from 5 different gene fusion transcripts in cell line RNA-seq data (three cell lines were used), when four RNA-seq libraries were pooled for each cell line for a total of 12 libraries and sequenced on a HiSeq 4000 lane. Only one cell line (K562) should have carried the BCR-ABL1 translocation, however, reads containing BCR-ABL1 were also found in data files for the other two cell lines due to index hopping. Costello et al also showed the variability of index hopping rate from pool to pool and flow cell to flow cell.

The present disclosure describes a method that can eliminate or mitigate significantly index hopping when large number of samples are analyzed in the same sequence run. It allows filtering out swapped reads from pooled samples. The methods of the present disclosure allow for mitigation of swapped reads caused by both multiplex PCR and sequencing-chemistry induced swaps. This is particularly crucial in clinical sequencing settings, single cell sequencing, detection and screening pathogens, or analysis of low allele fraction somatic variants where even low percentages of anomalous reads are unacceptable.

### SUMMARY OF THE INVENTION

In some embodiments, the present disclosure describes a method of eliminating or mitigating index hopping during amplification of at least one nucleic acid sample, comprising the steps of: for each sample, hybridizing a plurality of indexed target-specific primers with nucleic acid from the sample in the presence of indexed universal primers to form a plurality of test reactions in a single reaction container, wherein at least one indexed target-specific primer is configured to bind to at least one target nucleic acid sequence; subjecting each test reaction to amplification conditions to generate amplicons wherein each amplicon contains multiple indexes, wherein one index (A) at 5′ end of amplicon's at the Watson (forward) strand, one index (B) at 5′ end of amplicons at the Crick (reverse) strand, one index (X) next to target-specific primers at the Watson (forward) strand, one index (Y) next to target-specific primers at the Crick (reverse) strand; subjecting at least a portion of the amplicons to bead cleanup to form enriched amplicons; and sequencing the enriched amplicons, formed from each sample, by next-generation sequencing. In some embodiments, each index has a distinct sequence. In some embodiments, the amplicons are pooled immediately prior to bead cleanup.

In some embodiments, within a set of samples, the same X index (next to target-specific primers at the Watson strand) can be shared within more than one sample in the same set, and the same Y index (next to target-specific primers at the Crick strand) can be shared with more than one sample in the same set. The combination of X and Y indexes, however, needs to be unique for each sample within the same set. The same A index (at 5′end of amplicons at the Watson strand) can be shared within more than one sample in the same set, and the same B index (at 5′ end of amplicons at the Crick strand) can be shared with more than one sample in the same set. The combination of A and B indexes, however, must be unique for each sample within the same set. The same X and Y indexes can be used for samples in different sets (e.g., Set1, Set2, Set3, etc.) of samples pooled in the same sequencing run. However, each combination of X and Y indexes must be unique for each sample within each set. One the other hand, A and B indexes are NOT shared between different sets and must be unique to each set.

In another embodiment, the present disclosure may be performed in a two-step PCR, during amplification of at least one nucleic acid sample, comprising the steps of: for first-step amplification, for each sample, hybridizing a plurality of indexed target-specific primers with nucleic acid from the sample in a reaction container to form a test reaction, wherein at least one indexed target-specific primer is configured to bind to at least one target nucleic acid sequence; subjecting each test reaction to amplification conditions to generate amplicons wherein each amplicon contains two indexes, one index (X) next to target-specific primers at the Watson strand, one index (Y) next to next to target-specific primers at the Crick strand; performing the second-step amplification on at least a portion of nucleic acid amplicon products from the first-step amplification using indexed universal primers comprising two distinct indexes or, alternatively, subjecting each test reaction to amplification conditions to generate amplicons, wherein each amplicon contains indexes, wherein one index (A) at 5′ end of amplicons at the Watson strand, one index (B) at 5′ end of amplicons of Crick strand, one index (X) next to target-specific primers at the Watson strand, one index (Y) next to next to target-specific primers at the Crick strand; subjecting at least a portion of the amplicons to bead cleanup to form enriched amplicons; and sequencing the enriched amplicons, formed from each sample, by next-generation sequencing. In some embodiments, each final amplicon contains four (“quadruple”) combinatorial indexes. In some embodiments, each index has a distinct sequence. In some embodiments, the final amplicons are pooled immediately prior to bead cleanup.

In some embodiments, within a set of samples, the same X index can be shared within more than one sample in the same set, and the same Y index can be shared with more than one sample in the same set. The combination of X and Y indexes, however, needs to be unique in the same set. The same A index (at 5′ end of amplicons at the Watson strand) can be shared within more than one sample in the same set, and the same B index (at 5′ end of amplicons at the Crick strand) can be shared with more 11 than one sample in the same set. The same X and Y indexes can be used in different sets (e.g., S1, S2, S3, etc.) of samples pooled in the same sequencing run. However, each combination of X and Y indexes must be unique in each set. One the other hand, A and B indexes can NOT be shared between different sets and must be unique to each set.

In some embodiments, each indexed universal primer comprises: a universal priming portion at the 3′-end; a barcode/index portion in the middle; and a universal priming portion at the 5′-end. In some embodiments, each indexed target-specific primer comprises a universal priming portion, barcode/index portion and a specific sequence portion directed to a target nucleic acid sequence. In some embodiments, each sample is obtained from a subject, human, animal, plant, microbe, virus or an environmental source. In some embodiments, the target-specific primers comprise primers configured to amplify at least 10, 20, 50, 100, 200, 1,000 or more targets.

In some embodiments, the method further comprises the step of pooling the enriched amplicons from each sample prior to sequencing.

In some embodiments, the present disclosure describes a method of analyzing at least one sample from human, food, animal, plant, and pathogens, comprising the steps of: for each sample, hybridizing a plurality of indexed target-specific primers with nucleic acid from the sample in the presence of indexed universal primers to form a test reaction in a single reaction container, wherein at least one indexed target-specific primer is configured to bind to at least one target sequence, wherein each indexed universal primer comprises: a universal priming portion at the 3′-end; a barcode/index portion in the middle; and a universal priming portion at the 5′-end; and wherein each indexed target-specific primer comprises a universal priming portion, a barcode/index portion and a target-specific sequence portion; subjecting each test reaction to amplification conditions to generate amplicons with four indexes; pooling amplicons from each sample; subjecting at least a portion of the pooled amplicons generated from each sample to bead cleanup to form enriched amplicons; and sequencing the pooled and enriched amplicons, formed from each sample, by next-generation sequencing.

In some embodiments, the present disclosure describes a kit, comprising multiplex target-specific primers configured to bind to target sequences specific to: biological samples related to cancer, genetic disorders, forensic testing, allergens, microbial/viral species or pathogens, low-frequency somatic variant detection, ancient DNA, gene expression, cell-free DNA, ctDNA or any other nucleic acid biological applications.

In some embodiments, the present disclosure describes methods and compositions of amplifying selective target region(s) in a nucleic acid sample. In some embodiments, the method comprises the steps of: (1) contacting the nucleic acid sample with indexed target-specific primers in PCR reaction, in presence of indexed universal primers; and (2) allowing primer extension to generate target amplification products (amplicons) of different sizes wherein each amplicon contains quadruple (four) combinatorial indexes. In some embodiments, 4 out of 4 indexes in the amplicon comprise a distinct sequence or 3 out of 4 indexes in the amplicon comprise a distinct sequence. In some embodiments, the method comprises the step of determining the presence or absence of target amplification product. In some embodiments, the method comprises the step of establishing the sequence of the target amplification products. In some embodiments, less than 50, 40, 30, 20, 10, 5, 0.5, or 0.1% of the amplified products are primer-dimers or artifacts.

In some embodiments, the concentration of each indexed target-specific primer can be about 500, 250, 100, 80, 70, 50, 30, 10, 2, or 1 nM. In some embodiments, the GC content of the indexed target-specific primers can differ, and as an example it can be between 40% and 70%, or between 30% and 60% or 50% and 80%. In some embodiments, the melting temperature (Tm) of the indexed target-specific primers can be between 55° C. and 65° C., or 40° C. and 70° C., or 55° C. and 68° C. In some embodiments, the length of the indexed target-specific primers can be between 20 and 90 bases, 40 and 70 bases, 20 and 40 bases or 25 and 50 bases. In some embodiments, the 5′-region of the target-specific primer is a universal primer binding site that is not complementary or specific for any nucleic acid region in the sample. In some embodiments, the length of the target amplicons is between 50 and 500 bases; 90 and 350 bases, or 200 and 450 bases.

In various embodiments of any of the aspects of the present disclosure, the method of primer extension is based on the state-of-art polymerase chain reaction (PCR). In various embodiments, annealing time can be greater than 0.5, 1, 2, 5, 8, 10 or 15 minutes. In various embodiments, extension time can be greater than 0.5, 1, 2, 5, 8, 10 or 15 minutes.

In some embodiments, the method disclosed herein quantifies the copy number of the target sequence present in the sample.

In various embodiments of the present disclosure, the compatibility and non-compatibility score of the selected primers are calculated based on different factors of target amplicon GC content, target amplicon melting temperature, target amplicon heterozygosity rate, complementary rate of the candidate primer for the target region; candidate primer size, target amplicon size and amplification efficiency and off-target rate. The selected target-specific primers can hybridize to the nucleic acid target and selectively amplify the target regions. In various embodiments, the test sample is from a subject, individual, food, plant, animal, soil, environment or any nucleic acid subject that is suspected to have an infection or disease, or an increased risk for an infection or disease; and wherein one or more of the target nucleic acid comprise a sequence at the target region associated with an infection or disease or increased risk of an infection or disease. In some embodiments, the test sample is from a subject, individual, animal, soil, environment or nucleic acid subject not related to any diseases or infections. The profile of target regions can serve as identity mark for a subject, individual, animal, or other sample, in a way similar to fingerprint. In some embodiments, information can be used for disease screening, detection, disease management, pathogen surveillance, food recalls, outbreaks or pandemics.

In one embodiment, the method disclosed herein can be used to screen, detect and identify microbial and viral agents. In one embodiment, the method disclosed herein can be used to screen, detect, genotype, serotype, subtype and trace the source of infection (surveillance). In some embodiments, the candidate primers contact the nucleic acid sample; wherein the forward strand and reverse strand indexed target-specific primers hybridize to target nucleic acid regions (if present in the sample), where the nucleic acid sample may have microbial and/or viral organisms or is suspected to have microbial and/or viral organisms, amplifying a plurality of target nucleic acids in presence of indexed universal primers to generate amplicons containing four combinatorial indexes; subjecting the amplicons to next-generation sequencing; and analyzing the sequence data by software analysis. In some embodiments, the detected infections can be clinically actionable. In some embodiments, detected infections can be associated with drug resistance. In some embodiments, detection, identification, and quantitation of microbial and viral species, strains, and sub-strains can be related with disease. In some embodiments, the biological sample can be monitored for source of infection or surveillance.

In one aspect, the method and composition disclosed herein is designed to detect, identify, and quantify target nucleic acids in a sample that may contain microbial and viral organisms such as sexually transmitted infections (STI). In some embodiments the disclosed method comprises the steps of: (1) contacting the nucleic acid targets in a sample with primers, wherein indexed forward strand and indexed reverse strand target-specific primers hybridize to different nucleic acid target regions in the test reaction in the presence of indexed universal presence; (2) amplifying the target nucleic acids under optimal amplification conditions to generate amplicons containing quadruple combinatorial indexes; (3) sequencing the amplified products by NGS; and (4) analyzing and quantitatively measuring the generated sequence reads by a mapping-and-counting methodology.

In one embodiment, the method disclosed herein can be used to screen and analyze target regions of a genome for disease such as cancer or genetic disorder. In one embodiment, the method disclosed herein can be used for analyzing a genome for forensic DNA analysis based on the DNA profile such as short tandem repeat (STR) regions. In some embodiments, the method disclosed herein can be used for pharmacogenetics or drug resistance to detect the genetic variations that influence an individual's response to medication. In some embodiments, the candidate primers contact the nucleic acid sample; wherein the forward strand and reverse strand indexed target-specific primers hybridize to target regions in the presence of indexed universal primers, amplifying a plurality of target nucleic acids to generate amplicons with quadruple combinatorial indexes; subjecting the amplicons to next-generation sequencing; and analyzing the sequence data by software analysis. In some embodiments, the detected nucleic acid variations can be clinically actionable. In some embodiments, the biological sample can be monitored for prognosis.

In some embodiments, the nucleic acid sample comprises genomic nucleic acid. In some embodiments, the sample comprises nucleic acid molecules obtained from food, vegetables, produce, plants, soil, spoilage, water, environment, food production facilities or any nucleic acid subject. In some embodiments, the sample comprises nucleic acid molecules obtained from urine, tissue, saliva, biopsies, sputum, swabs, surgical resections, cervical swabs, tumor tissue, fine needle aspiration (FNA), scrapings, swabs, mucus, semen, other non-restricting clinical or laboratory obtained samples.

In another aspect, the present disclosure is directed to kits comprising indexed target-specific primers for amplifying target regions of interest in a sample.

In some embodiments, the disclosed method comprises the steps of: performing multiplex barcoding amplification generating amplicons containing four combinatorial indexes, and sequencing the resulting amplicons by NGS. In certain embodiments, the samples are obtained from subjects with single or multiple co-infections. In some embodiments, the method's analytical sensitivity is 10 copies for each microbial and viral species in a sample; the highly multiplex PCR amplifies 20, 50, 100, 200, 500, 1,000 or more targets with minimal primer-primer interactions. In some embodiments, the method comprises the step of performing single-reaction, single-step barcoding multiplex PCR. In some embodiments, the method can analyze 5, 10, 20, 50, 100, 200, 500, 1,000, 2,000, 5,000, 10,000 or more samples by a single NGS sequencing run.

In some embodiments, the disclosure relates to methods, compositions, and kits for application of multiplex target amplification and target enrichment prior to downstream analysis such as next generation sequencing. The method relies on using a plurality of indexed target-specific primers in presence of indexed universal primers and target enrichment amplification in a DNA sample that is suspected to have disease (cancer or genetic disorder), drug resistance, forensics information, or microbial and viral pathogens. The target-specific primers amplify the target nucleic acids under optimal conditions in presence of amplification reagents such as polymerase and dNTPs to at least amplify one or more nucleic acid targets of interest.

In some embodiments of the disclosed method, the primer design methodology selects the candidate target-specific primers based on steps of: (1) extracting genomic sequences; (2) designing a set of target-specific forward strand and reverse strand target-specific primers for target sequences with proper GC content, Tm, and varying distances from each targeted region; (3) for each primer, searching target genome sequences for off-target matches; filter primers and keep those primers that pass the off-target threshold; (4) searching the 3′-end portion of each primer for complementary matches with primer sequences of the set; filter primers progressively where the primer with its 3′-end having most complementary matches is removed first; and (5) synthesizing primers and running the entire wet-lab experiment using next-generation sequencing; calibrate the performance of each primer and filter out primers of undesired performance. In some embodiments, the primer selection procedure steps 2 to 4 and steps 2 to 5 are repeated until each target sequence is covered by at least one forward strand target-specific primer and one reverse strand target-specific primer in the primer set.

In various embodiments of any of the aspects of this disclosure, the methods and compositions feature multiplex barcoding amplification and target enrichment of target nucleic acid regions with indexed target-specific primers in presence of indexed universal primers in a single reaction. In some embodiments, the disclosed method comprises the steps of: (1) contacting indexed target-specific primers with target nucleic acid sequences in presence of indexed universal primers and hybridizing to target nucleic acid sequences in the sample; (2) subjecting the test reaction to amplification under optimal amplification conditions and generating amplicons containing quadruple combinatorial indexes; (3) pooling together the amplified products from each individual sample; (4) subjecting a portion of the pooled amplified products to bead cleanup to remove unconsumed. primers and primer-dimers and create enriched amplified products; (5) subjecting a portion of enriched amplified products to standard normalization and quantification; and (6) sequencing the amplicon by next-generation sequencing.

In one embodiment, the indexed universal primers comprise: a) a universal priming portion at the 3′-end; b) a barcode/index portion in the middle; and c) a universal priming portion at the 5′-end (FIG. 1.). In one embodiment, each indexed target-specific primer comprises a universal priming portion, a barcode/index portion, a specific sequence portion directed to target nucleic acid sequence (FIG. 1).

In some embodiments, the composition comprises a plurality of indexed target-specific primers wherein at least one target-specific primer is at least 90% identical to any one of the nucleic acid targets. In some embodiments, the composition comprises a plurality of target-specific primers having a sequence identity of at least 91%, 92%, 93%, 94%, 95%, 96%, 97%, 98% or 99% to the nucleic acid targets in the sample.

In some embodiments, the disclosure relates to a composition comprising a plurality of indexed target-specific primers wherein the sequence complementary to target nucleic acid of interest is about 15 to 40 bases in length.

In some embodiments, the disclosure relates to a composition of pre-calculated design of indexed target primers that generate minimal cross-hybridization or primer-primer interactions with other target-specific primers in the composition. In some embodiments, the primers in the composition are designed to avoid non-specific priming that can lead to non-specific amplifications. In some embodiments, the amplification conditions such as annealing temperature, annealing duration and primer concentrations can be adjusted to minimize amplification artifacts such as primer-dimers.

In some embodiments, the disclosure relates to a method or composition comprising a plurality of indexed target-specific primers having minimal cross-hybridization to non-specific sequences present in the sample. In some embodiments, such cross-hybridization to non-specific targets can be monitored and evaluated by downstream analysis such as next generation sequencing.

In some embodiments, the disclosure relates to a method or composition comprising a plurality of indexed target primers having minimal self-complementary structure. In some embodiments, the composition comprises at least one target-specific primer that do not form a secondary structure, such as hairpins or loops. In some embodiments, the composition comprises a plurality of target-specific primers that the majority, or potentially all the target-specific primers do not form secondary structures such as hairpins and loops.

In some embodiments, the target nucleic acid is obtained from a subject. In some embodiments, the sample comprises proteins, cells, fluids, biological fluids, preservatives, and/or other substances. In certain embodiments, the sample originates from urine, tissue, saliva, biopsies, sputum, swabs, surgical resections, cervical swabs, tumor tissue, fine needle aspiration (FNA), scrapings, swabs, mucus, semen, other non-restricting clinical or laboratory obtained samples.

In some embodiments, the target amplification products are sequenced by next generation sequencing on current state-of-art next-generation sequencing technologies or platforms. In some embodiments, the disclosed method is not limited to these next-generation sequencing technologies examples and can be applied to new sequencing innovations.

In certain embodiments, the foregoing methods may be performed at multiple time points.

### BRIEF DESCRIPTION OF THE DRAWINGS

The disclosure can be better understood with reference to the following drawings. The elements of the drawings are not necessarily to scale relative to each other, emphasis instead being placed upon clearly illustrating the principles of the disclosure. Furthermore, like reference numerals designate corresponding parts throughout the several views.

FIGS. 1A-1D depict an illustration of: forward indexed target-specific primer comprising of read 1 sequencing (universal) portion, X index portion and target-specific sequence portion (FIG. 1A); reverse indexed target-specific primer comprising of read 2 sequencing (universal) portion, Y index portion and target-specific sequence portion (FIG. 1B); indexed universal primer comprises of a universal portion (P5 tail), A index portion and read 1 sequencing (universal) portion (FIG. 1C); indexed universal primer comprises of a universal portion (P7 tail), B index portion and read 2 sequencing (universal) portion (FIG. 1D).

FIGS. 2A-2D depict an illustration of the process in a single tube and single amplification reaction where forward and reverse indexed target-specific primers in presence of indexed universal primers amplify the nucleic acid targets (FIGS. 2A-2C). After amplification, each amplicon is labeled with four indexes (FIG. 2D).

FIG. 3 depicts an illustration of an amplicon after amplification with the four indexes as well as the Illumina@ sequencing procedure. Four sequencing reads are generated in a sequencing run. Read 1 is comprised of X index and target sequence. Read 2 is comprised of Y index and target sequence. The A index and B index are generated with independent reads.

FIG. 4 illustrates indexing primers layout of a 96-well plate.

FIG. 5 illustrates same forward-and reverse-primer sets paired with a second index set of P5-primers with X indexes and P7-primers with Y indexes.

FIG. 6 illustrates same forward-and reverse-primer sets are paired with a third index set of P5-primers with X indexes and P7-primers with Y indexes.

FIG. 7 illustrates the single tube multiplexed barcoding amplification workflow.

FIGS. 8A and 8B shows two tables listing read counts, FIG. 8A is a table that lists the reads count for three targets, wherein the numbers for permissible combinations are highlighted with grey background and there are 57 unintended reads. FIG. 8B is a table that shows 16 unintended reads after removing reads with impermissible combinations of indexes.

FIG. 9 shows a table listing 5 sequencing runs with a total of 480 samples (96 samples per sequencing run) amplified by an HPV-STI assay, where 45.7% to 66.6% false positive targets for each sequencing run were removed by combinatorial quadruple indexing method.

### DETAILED DESCRIPTION

The present disclosure relates to a method of combinatory indexing, where amplification and barcoding occur simultaneously in the same PCR reaction (end library product) where the amplicons contain four combinatorial indexes. The four-indexed amplicons are further analyzed by systems such as next-generation sequencing. The present invention has a universal approach and can be applied for nucleic acid-based biological application such as: (1) screening, detection and identification of bacteria/viral/parasitic/fungal and microbiome organisms; (2) cancer/genetic disease nucleic acid sequence analysis; (3) pharmacogenetics and companion diagnostics for selecting the right treatment as well as prognosis; (4) drug resistance for applications such as antimicrobial therapy selection, surveillance and epidemiology, and personalized medicine; (5) forensic DNA analysis for DNA profiling; (6) allergens; (7) low-frequency somatic variant detection, ancient DNA, gene expression, cell-free DNA, ctDNA and/or (8) or any nucleic acid-based biological application that uses the four-index multiplex barcoding amplification. The present invention provides methods, compositions, kits, systems, and instruments that will allow such target enrichment.

Improvements in NGS technology have greatly increased sequencing speed and data output, resulting in the massive sample throughput of current sequencing platforms. A key to utilizing this increased capacity is multiplexing, which may, as under the disclosed methodology, add unique sequences, called indexes, to each DNA fragment during library preparation. This allows large numbers of libraries to be pooled and sequenced simultaneously during a single sequencing run. With multiplexing, the potential for index hopping is present regardless of the library prep method or sequencing system(s) used. Index hopping may result in assignment of sequencing reads to the wrong index during demultiplexing, leading to misassignment. Libraries with higher levels of free adapters will see higher levels of index hopping. Because oligonucleotide/primer synthesis is intrinsically error-prone with error frequencies rated at 1-5% per base, it is desirable to maximize the difference between two indexes. Given that indexes are used to label samples and do not contribute to interrogate the target sequence in a sample, indexes are consequently designed with minimal length and with maximal differences among them when used in the same multiplex pool. For these reasons, indexes are normally designed to be 6-10 bases long, with 3-5 bases difference among them. With such constraints and other considerations such as avoiding low complexity, avoiding long-stretch polymers, maintaining balanced GC content and so on, only limited number of indexes can be designed.

To label different samples in a multiplex pool, each sample must be labeled with a unique index in a process named single unique indexing. For instance, to multiplex 100 samples, 100 unique indexes are needed. To multiplex 10,000 samples, 10,000 unique indexes are needed. Besides the burden of synthesis and evaluation of a large number of indexes, operations with so many indexes are prone to cross-contamination, given that many containers with indexes need to be opened and closed. Moreover, it is a challenge to streamline a functional, cost-efficient and practical workflow.

Unique dual indexes consist of two distinct indexes or barcode sequences that are added to each DNA fragment. These indexes allow for multiplexing, enabling multiple samples to be sequenced together in a single sequencing run while maintaining the ability to identify and separate the data for each individual sample during analysis. Each DNA fragment from every sample is tagged with a unique combination of dual indexes. This way, even if all the samples are sequenced together in a pooled manner, the resulting sequencing data can be demultiplexed using these unique dual indexes to assign each read to its original source, ensuring a more accurate analysis and interpretation of the sequencing data for each sample. Unique dual indexes allow to increase the number of samples sequenced per run and reduce per-sample cost compared to other indexing strategies. With just one unique dual index plate, a user can pool 96 samples together. In addition to unique dual indexes, another strategy for dual index sequencing is to use combinatorial dual indexes, which allows sequences to be repeated across rows and columns of a well plate.

By using a combinatorial dual indexing approach, fewer number of indexes can be used to label a larger number of samples. For instance, 10 indexes can be used to label one end of DNA fragments, and 10 indexes can used to label the other end of DNA fragments. In total, 20 indexes can be used to label 100 (10×10) samples. Similarly, 100 indexes can be used to label one end of DNA fragments, and 100 indexes can be used to label the other end of DNA fragments. In total 200combinatorial dual indexes can label 10,000(100×100) samples. The operational complexity of the combinatorial dual indexing approach is significantly lower than single unique indexing or dual unique indexing methods.

In contrast to conventional combinatorial dual indexing, unique dual indexing has distinct, unrelated and unique index sequences (96 unique A indexes and 96 unique B indexes for a 96-well plate) that mitigates misassigned reads. However, this indexing strategy is not preferable for enhanced multiplexity capacity and large sample scales. On the other hand, in conventional combinatorial dual indexing, there is a limit to 8 unique dual pairs in a 96-well plate, where the majority of amplicons share common indexes on the A and B index ends. The conventional combinatorial dual indexing is suitable for enhanced multiplexity capacity and larger sample scales but generates significantly higher contamination misassignments.

In practical conditions, operation with many indexes is prone to cross-contamination, as there are many containers with indexes that are opened and closed. Therefore, even if the unique dual indexing approach could lower the risk of index hopping in theory, the operation of this approach may increase the risk of cross-contamination.

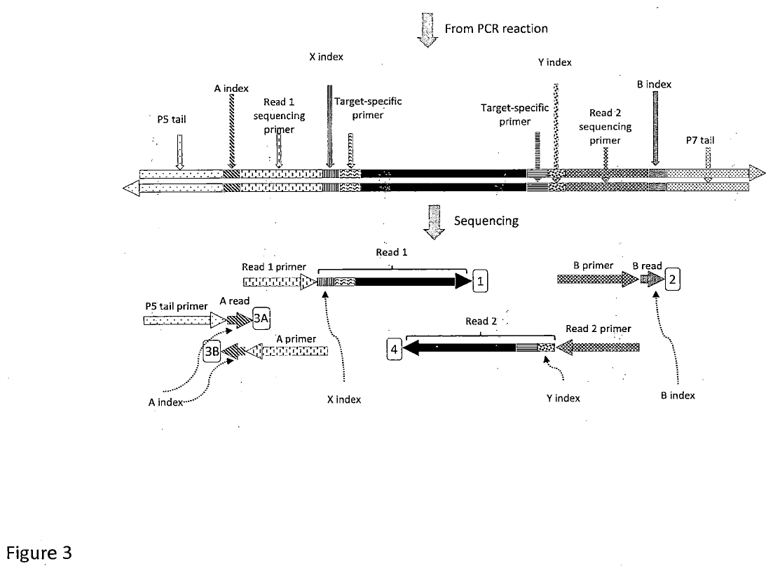

Nevertheless, the conventional combinatorial dual indexing approach increases the risk of index hopping because for the samples share an identical index at one end, and the indexes are different at the other end. As disclosed herein, a way to resolve this and mitigate the risk of index hopping was developed by the use of Combinatorial Quadruple Indexing (CQI), whereby each end of a DNA fragment/amplicon is tagged with two indexes or a total of 4 combinatorial indexes on both ends (FIG: 2 and FIG. 3).

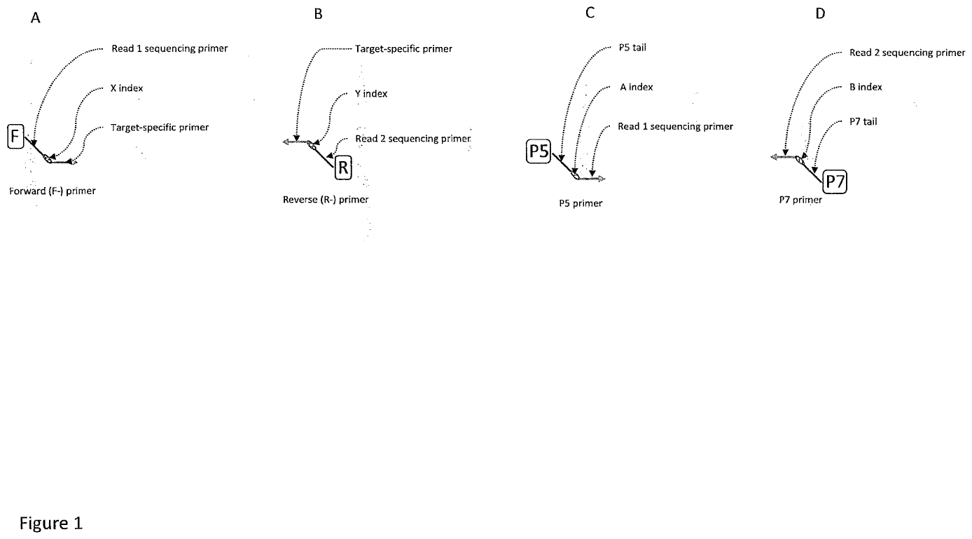

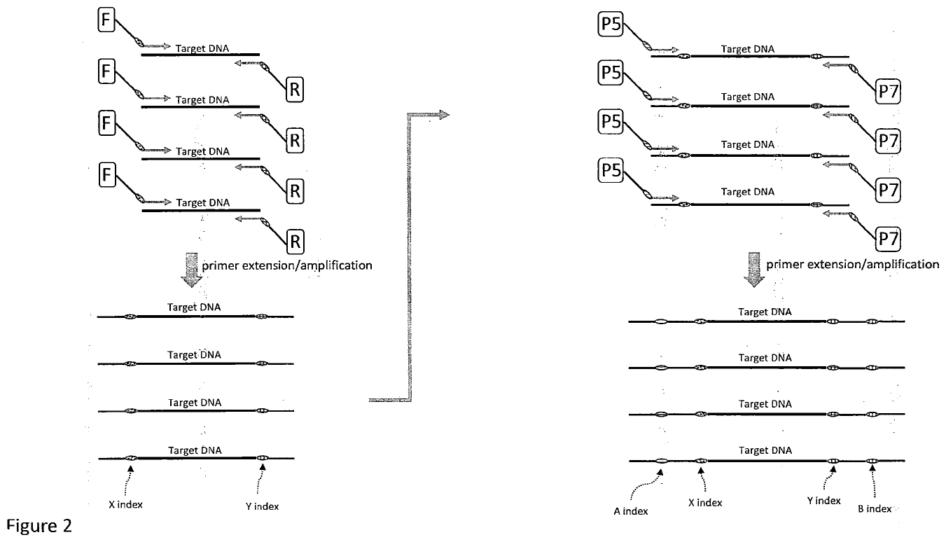

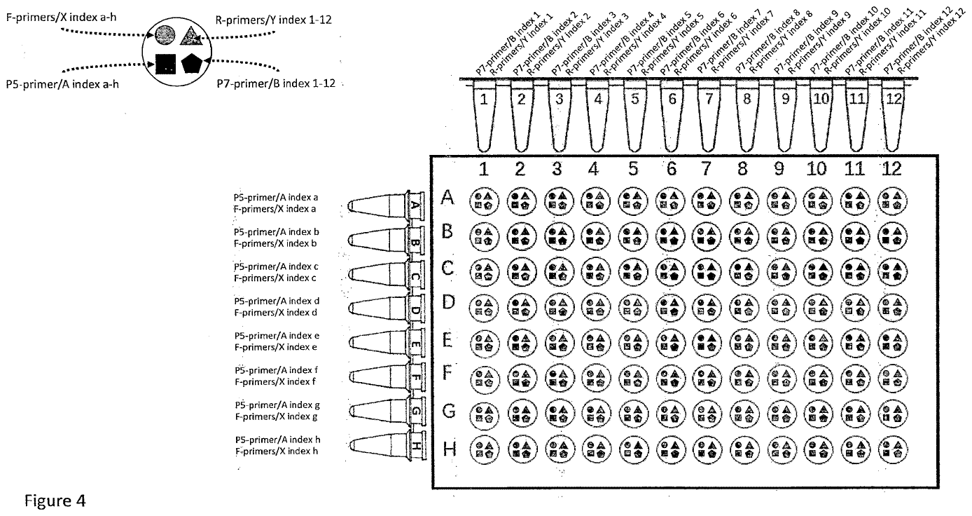

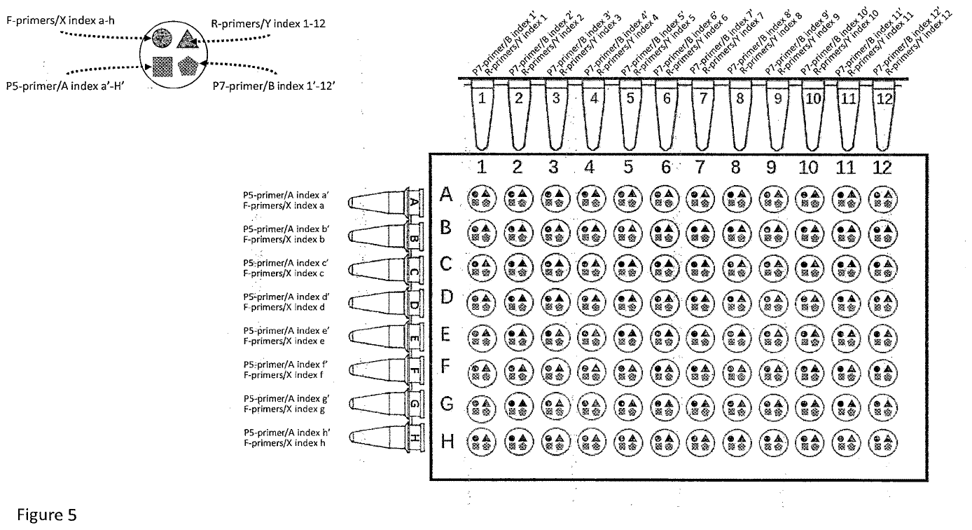

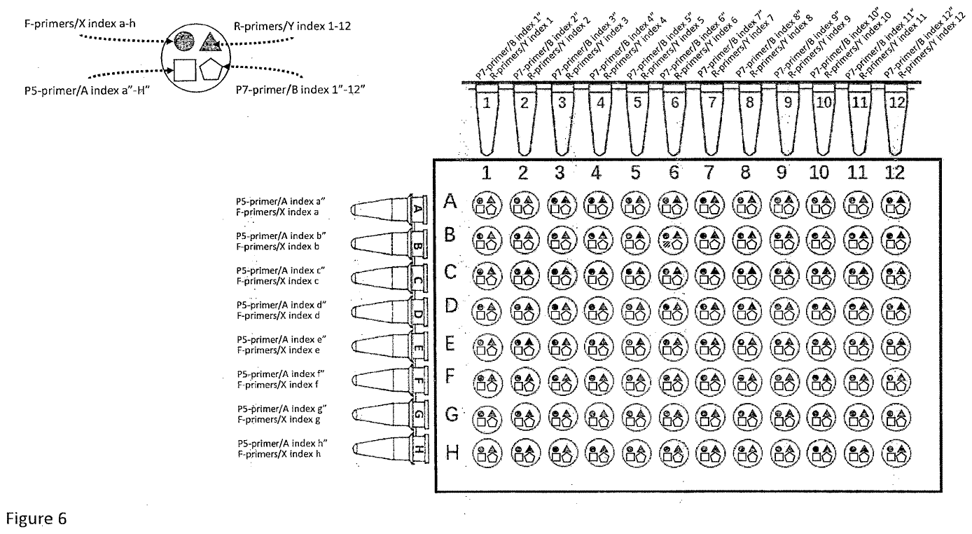

The Combinatorial Quadruple Indexing or “CQI” mitigates or eliminates the index-switching risk with lower operational complexity than that of combinatorial dual indexing approach. In the CQI approach, targets are first labeled with indexed forward primers and indexed reverse primers in early stage of PCR reaction (FIGS. 2A and 2B). These early-stage products are then amplified by indexed universal primers (P5-primer and P7-primer) to hybridize and amplify early staged amplicons to generate quadruple indexed amplicons (FIGS. 2C and 2D). Each indexed forward (F) primer is comprised of read 1 sequencing primer, X index, and target-specific primer (FIG. 1A). Each indexed reverse primer (R) is comprised of read 2 sequencing primer, Y index, and target-specific primer (FIG. 1B). The indexed universal primer (P5) primer is comprised of P5 tail, A index, and read 1 sequencing primer (FIG. 1C). The indexed universal primer (P7) is comprised of P7 tail, B index, and read 2 sequencing primer (FIG. ID). After amplification, each DNA fragment is labeled with four combinatorial indexes in total (FIG. 2). The A index and B index are sequenced as independent reads. X and Y indexes are sequenced at the beginning of read 1 and read 2 (FIG. 3). The A index and X index in forward primers are combined as two unique indexes (forward indexes). The B index and the Y index in reverse primers are combined as two unique indexes (reverse indexes). The end amplicon product is labeled with quadruple or four combinatorial indexes. It is notable that a single index switch cannot convert a permissible combination to another permissible combination. Together, the forward index and reverse index act as quadruple indexes on the same amplicon with the capacity to mitigate significantly the risk of index hopping. The Combinatorial Quadruple Indexing approach is based on two sets of combinatorial indexing that mitigates or eliminates index hopping, uses significantly fewer number of indexes in contrast to unique dual indexing, greatly enhances the multiplexity capacity, thus applicable for barcoding larger number of samples and achieves the same level of specificity as unique dual indexing. CQI is user-friendly, cost-, time- and labor-efficient in laboratory settings. Examples of quadruple indexing primer layouts using 96-well plates are shown in FIGS. 4-6.

The examples, applications, descriptions and content disclosed herein are exemplary and explanatory, and are non-limiting and non-restrictive in any way.

The present disclosure comprises of a one-step, single-tube four-index barcoding multiplex amplification step that can be applied for a wide range of biological applications. In the PCR step, amplification and four-index barcoding of nucleic acid targets occur simultaneously in the same reaction and is then followed by NGS and data analysis.

In one aspect, the method and composition disclosed herein is designed to analyze target nucleic acids in a sample that is analyzed for disease (cancer/genetic disorders), drug resistance, genetic profile, forensics or microbial and viral organisms (bacteria, fungi, parasites or viruses), allergen and other biological applications. In some embodiments the disclosed method comprises the steps of: (1) contacting a set of nucleic acid targets in a sample with primers, wherein indexed forward strand and reverse strand target-specific primers hybridize to target in the presence of indexed universal primers in the test reaction; (2) amplifying the target nucleic acids under optimal amplification conditions to determine presence or absence of target nucleic acid; (3) sequencing the four-indexed amplified products by NGS; and (4) analyzing the generated sequence reads.

Developing highly multiplex amplification methods for nucleic acid sample with accurate and high copy number sensitivity remains a challenge in the art. The present disclosure relates to an NGS-based assay that combines balanced target-specific multiplex amplification and sensitive copy number quantification, and balanced sequencing reads of each target while the four indexed barcoding of each target amplicon occurs simultaneously in the amplification step.

All scientific terms used herein have the same meaning as commonly used and understood by one of ordinary skill in the art. Examples, materials, methods, figures and tables are illustrative only and not intended to be limiting.

As used herein, “amplification conditions” means conditions suitable for amplification using polymerase chain reaction. The polymerase chain reaction can be multiplex PCR. Amplification conditions include, but are not limited to, the examples provided in Examples 1-6 disclosed herein.

As used herein, “indexed universal primer” means a universal primer comprising a barcode/index sequence and at least one universal sequence. See, e.g., FIGS. 1C and 1D.

As used herein, “bead cleanup” means the use of bead-based purification wherein beads are configured to bind to one or more targets. As known to those of skill in the art, bead cleanup may use positive selection (i.e., the bead is configured to capture the target of interest) or negative selection (i.e., the bead is configured not to capture the target of interest). Various may be used, as known in the art, such as streptavidin beads or magnetic beads.

As used herein, “compatibility score” means a score for a potential forward strand target-specific primer or reverse strand target-specific primer that is calculated based on different factors of target amplicon GC content, target amplicon melting temperature, target amplicon heterozygosity rate, complementary rate of the candidate primer for the target region; candidate primer size, target amplicon size, primer-primer interactions and amplification efficiency and off-target rate.

As used herein, “dsDNA” means double stranded DNA.

As used herein, “environmental source” means any potential location in a natural and/or man-made environment from which a sample can be taken. Environmental sources include but are not limited to: water sources such as oceans, lakes, ponds, rivers and streams; sources of soil such as soil, sand, internal or external dust; sources of gas, such as air.

As used herein, “FNA” means fine needle aspiration.

As used herein, “forward strand” means one (single) strand of a dsDNA sample.

As used herein, “indexed forward strand target-specific primer” or “indexed forward primer” means a primer configured to bind to a target sequence on the forward strand, wherein the primer is configured to introduce an index in the resulting amplicon See, e.g., FIG. 1A.

As used herein, “GC content” means guanine-cytosine content.

As used herein, “locus” means a specific physical position or location on the genome where a particular gene or genetic marker is located.

As used herein, “microbial and viral surveillance” means systematic monitoring and tracking of bacterial and viral pathogens in populations or specific geographical areas. It involves the collection, analysis, and reporting of data related to the occurrence, distribution, and characteristics of bacterial and viral infections.

As used herein, “index”, “index” means a short, unique nucleic acid sequence that is added to individual DNA fragments before sequencing in order to distinguish and identify different samples or DNA fragments within a single sequencing run.

As used herein, “index hopping”, “index hopping”, “index bleeding” or “barcode hopping/switching/swapping” means when index (barcode) sequences initially assigned to a specific sample are incorrectly assigned to other sample(s) in a pool of samples. The result of index hopping is the incorrect assignment of sample reads (DNA fragments) in pooled samples.

As used herein, “multiplex barcoding amplification” means multiplex target amplification where amplification and indexing/barcoding of each target occurs simultaneously in the PCR reaction.

As used herein, “NGS” means next-generation sequencing.

As used herein, “PCR” means polymerase chain reaction.

As used herein, “reverse strand” means a second (single) strand of a dsDNA sample that is complementary to the forward strand.

As used herein, “indexed reverse strand target-specific primer” or “indexed reverse primer” means a primer configured to bind to a target sequence on the reverse strand, wherein the primer is configured to introduce an index in the resulting amplicon See, e.g., FIG. 1B. As used herein, “sample” means a specimen or a preparation in the field to which the present invention pertains. Samples may be obtained from various sources, such as subjects, food, plants, and environmental sources. In case where the term is used in the present specification with respect to a subject, for example, the “sample” means a “biological sample” or an equivalent thereof. The “biological sample” means any preparation obtained from a biological material (e.g., individual, liquid, body fluid, cell line, cultured tissue or tissue segment) serving as a source. Examples of the “biological sample” include body fluids (e.g., blood, saliva, dental plaque, blood serum, blood plasma, urine, synovia, and cerebrospinal fluid) and tissue sources.

As used herein, “species” means a group of microorganisms that share similar genetic and phenotypic characteristics.

As used herein, “subject” means an animal, preferably a mammal, and most preferably a human.

As used herein, “target-specific primer” means a primer configured to bind to a specific target.

As used herein, “type” in microbiology means to refer to the strain or specific type of a microorganism. In virology, it means classification of viruses based on their genetic and antigenic characteristics.

As used herein, “type-specific primer” means a primer configured to bind to a target that is specific to a particular microbial or viral genome.

As used herein, “universal sequence” means a sequence configured to be targeted by a universal sequence primer.

As used herein, “unique dual indexing” means short, distinct DNA sequences that are attached to individual DNA fragments before sequencing. They consist of two distinct indexes or barcode sequences that are tagged to each DNA fragment. These indexes allow for multiplexing, enabling multiple samples to be sequenced together in a single sequencing run while maintaining the ability to identify and separate the data for each individual sample during analysis.

As used herein, “Combinatorial Dual Indexing” means multiple indexing sequences, often in pairs or sets, are used to label and differentiate DNA samples before they undergo high-throughput sequencing. Each set of indexes, typically consisting of two separate sequences (dual indexes). With combinatorial dual indexes, sequences are repeated across rows and columns of a well plate. By using multiple sets of such indexes, it increases multiplexity capacity.

The present disclosure relates to selective amplification of a set of target sequences by multiplex barcoding amplification and further analysis by next-generation sequencing. The disclosure has universal approach for a wide range of nucleic acid-based biological applications.

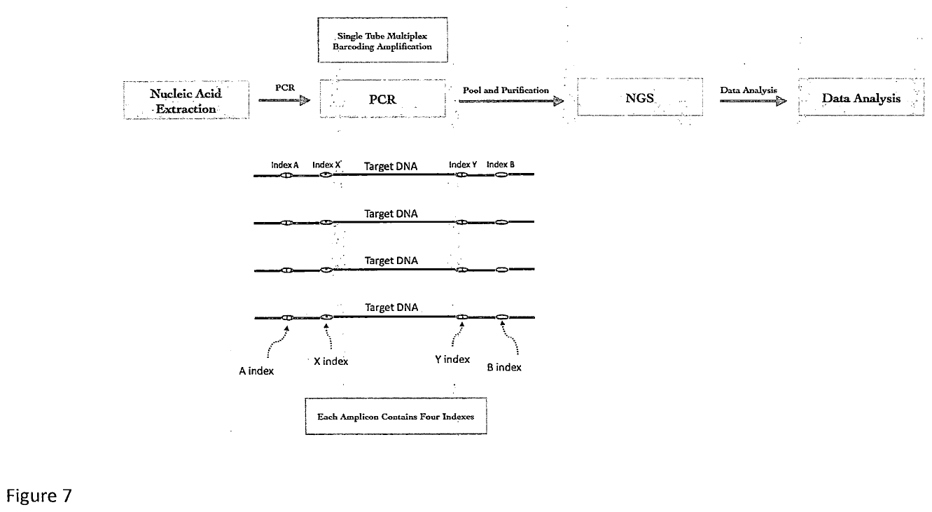

The disclosed method offers many advantages over conventional methodologies including, but not limited to: (1) greatly enhancing multiplexity capacity, by allowing barcoding of large number of samples with fewer number of indexes; (2) at the same time, mitigating or eliminating index hopping rate down to the same level as the unique dual indexing, (3) cost-and time-efficient due to the large coding capacity of combinatory indexing approach, where N is forward indexes and M is reverse indexes, the CQI could generate unique labels for N×M samples, which is significantly cost-saving in primer synthesis and time-and labor-efficient in laboratory operations and workflows in contrast to unique dual indexing; (4) user-friendly and cost efficient assay design and assay development as only one set of internal combinatory indexes (indexes close to gene-specific primers) and one set of external combinatory indexes (indexes close to the ends of amplicons) are necessary for one set of samples. See, e.g., FIG. 7.

Herein described is a method of multiplex barcoding amplification and target enrichment, which are analyzed by NGS (e.g., FIG. 7). In various embodiments of any of the aspects of the present disclosure, the methods and compositions feature multiplex barcoding amplification and target enrichment of target nucleic acid regions of genomic material suitable for the assessment of cancer, genetic disorders, drug resistance, forensics, allergens, microbial and viral organisms and other biological applications.

In some embodiments, the present disclosure can be applied for detection, identification and typing of microbial and viral pathogens such as sexually transmitted infections and HPV. In some embodiments, the disclosed method comprises the steps of: (1) contacting indexed target-specific primers with a set of target nucleic acid sequences in the presence of indexed universal primers and hybridizing to target nucleic acid sequences in the sample; (2) subjecting the test reaction to amplification under optimal amplification conditions to generate amplicons with quadruple combinatorial indexes; (3) pooling together the amplified products from each individual or subject sample; (4) subjecting a portion of the pooled amplified products to bead cleanup to remove possible unconsumed primers and primer-dimers to create enriched amplified products; (5) subjecting a portion of enriched amplified products to standard normalization and quantification; and (6) sequencing the amplicon by next-generation sequencing. See, e.g., FIG. 7.

In some embodiments, the present disclosure can be applied for nucleic acid sequence analysis of target regions for cancer, genetic disorders, forensics, pharmacogenetics, and/or drug resistance. In some embodiments, the disclosed method comprises the steps of: (1) contacting indexed target-specific primers to target nucleic acid sequences in a sample in the presence of indexed universal primers and hybridizing to target nucleic acid sequences in the sample; (2) subjecting the test reaction to amplification under optimal amplification conditions generating amplicons with quadruple combinatorial indexes; (3) pooling together the amplified products from each individual or subject sample; (4) subjecting a portion of the pooled amplified products to bead cleanup to remove possible unconsumed primers and primer-dimers to create enriched amplified products; (5) subjecting a portion of enriched amplified products to standard normalization and quantification; and (6) sequencing the amplicon by next-generation sequencing. See, e.g., FIG. 7.

In one embodiment, the barcoded universal primers comprise: (a) a universal priming portion at the 3′-end; (b) a barcode/index portion in the middle; and (c) a universal priming portion at the 5′-end (FIG. 1C and ID). In some embodiments, each indexed target-specific primer comprises a universal priming portion, a barcode/index portion, and a specific sequence portion directed to a target nucleic acid sequence.

In some embodiments, the disclosed method utilizes one round of multiplex barcoding PCR in one single test reaction for each subject, which eliminates or minimizes index hopping and cross-contamination and extra steps in the workflow. In contrast, conventional methods that use more than one round of PCR are vulnerable to DNA cross-contamination, longer workflow duration, and automation challenges.

In some embodiments, the disclosed method comprises the use of a quadruple combinatorial indexing for each amplicon in a single PCR reaction, wherein the amplicon is barcoded by 1) indexed forward and indexed reverse target-specific primers and 2) indexed universal primers, eliminating and minimizing index hopping and cross-contamination. In some embodiments, each indexed universal primer comprises: a universal priming portion at the 3′-end; an index/barcode portion in the middle; and a universal priming portion at the 5′-end. In some embodiments, each indexed target-specific primer comprises a universal priming portion, a barcode/index portion and a specific sequence portion directed to a target nucleic acid sequence. In some embodiments, each sample is obtained from a subject, human, animal, plant, microbe or an environmental source. In some embodiments, the target-specific primers comprise primers configured to amplify at least 10, 20, 50, 100, 200, 1,000 or more targets.

In some embodiments, the disclosed method comprises the use of next-generation sequencing for screening, detection, identification, and quantification of nucleic acid targets. In certain embodiments, target nucleic acid sequence's are amplified and sequenced to detect, identify and type pathogens such as sexually transmitted infections. In some embodiments, the disclosed method comprises the use of next-generation sequencing for analyzing target nucleic acid regions of genomic material of samples related to cancer/genetic disorders, drug resistance, forensics, allergens and other biological applications.

In some embodiments, the amplification conditions such number of cycles, annealing temperature, annealing duration, extension temperature and extension duration are adjusted to optimal conditions for amplification. In some embodiments, number of cycles, the amplification conditions such annealing temperature, annealing duration, extension temperature and extension duration are adjusted to optimal conditions for amplification based on the commercial DNA polymerase instructions.

In some embodiments, the nucleic acid sample comprises genomic DNA or RNA. In another embodiment, the sample comprises nucleic acid molecules obtained fresh produce, food, imported food, food production facilities, farms, fresh produce, animal farms, water, spoilage, soil and environment. In another embodiment, the sample comprises nucleic acid molecules obtained from swab or brush. In some embodiments, the sample comprises nucleic acid molecules obtained from saliva. In some embodiments, the sample comprises nucleic acid molecules obtained from urine, tissue, saliva, biopsies, sputum, swabs, formalin-fixed paraffin-embedded material (FFPE), surgical resections, cervical swabs, tumor tissue, fine needle aspiration (FNA), scrapings, swabs, mucus, urine, semen, and other non-restricting clinical or laboratory obtained samples.

In some embodiments, the nucleic acid sample obtained can be from an animal such as a human or mammalian subjects. In another embodiment, the nucleic acid sample obtained can be from a non-mammalian subject such as bacteria, parasites, virus, fungi, and plant.

In some embodiments, the disclosure relates to target amplification of at least one target sequence from a biological sample in a normal or diseased subject. In some embodiments, the disclosure relates to the specific and selective target amplification of at least one target sequence in the nucleic acid sample.

In some embodiments, the indexed target-specific primers comprise a plurality of primers that are designed to amplify microbial and viral target nucleic acid sequences. In some embodiments, the target-specific primers comprise a plurality of indexed target-specific primers that are designed to amplify selectively target nucleic acid sequences of genomic material of samples related to cancer, genetic disorders, drug resistance, forensics, allergens and/or other biological applications.

The amplification range differs due the size of fragments and positions of primers on the nucleic acid fragment and the size can vary in the range. In some embodiments, the target-specific primers comprise a plurality of primer that are selectively designed to amplify target nucleic acid sequences, where the amplified target nucleic acid sequences can vary in length from one another by no more than 90%, no more than 70%, no more than 50%, no more than 25% or no more than 10%.

In some embodiments, the disclosed method relates to target enrichment by multiplex barcoding target-specific PCR, which comprises the steps of contacting the nucleic acid targets with a plurality of indexed target-specific primers in the presence of indexed universal primers and PCR reagents such as DNA polymerase, dNTPs and reaction buffer; given the optimal conditions of temperature and time for denaturation, annealing and extension, the primers hybridize to complementary target nucleic acid sequences and are extended. In some embodiments, the amplification steps can be performed in any order. In some embodiments, amplification steps, purification steps and cleanup steps can be added or removed upon optimization for optimal multiplex target amplification for downstream processes.

In some embodiments, the described method uses PCR and DNA polymerase as one of the components in the reaction. In some embodiments, there are a wide selection of DNA polymerases, which feature different characteristics such as thermostability, fidelity, processivity and Hot Start. The method can use a DNA polymerase with one or more of these features depending on the application. In some embodiments, the concentration of DNA polymerase for multiplex PCR can be higher than single-plex PCR.

In some embodiments, the method disclosed herein uses amplification of target nucleic acid sequences using multiplex polymerase chain reaction, wherein more than one target sequence is amplified in a test reaction. In some embodiments, the amount of nucleic acid sample needed for multiplex amplification can be about 0.1 ng. In some embodiments, the amount of nucleic acid material can be about 1 ng, 5 ng, 10 ng, 50 ng, 100 ng or 200 ng.

In some embodiments, the disclosed method uses amplification of target nucleic acid sequences using multiplex polymerase chain reaction, wherein more than one target sequence is amplified in a test reaction. The state-of-art polymerase chain reaction is performed on a thermocycler and each cycle of PCR comprises of denaturation, annealing and extension. Each cycle of PCR comprises at least denaturation step, one annealing step and one extension step for extension of nucleic acids. In some embodiments, annealing and extension can be merged. In some embodiments, the method disclosed herein comprises 25 to 35 cycles of PCR. Each cycle or set of cycles can have different durations and temperatures, for example the annealing step can have incremental increases and decreases in temperature and duration, or the extension step can have incremental increases and decreases in temperature and duration. In some embodiments, duration can have decreases or increases in 5 seconds, 10 seconds, 30 seconds, 1 minute, 2 minutes, 4 minutes, 8 minutes, or greater increments. In some embodiments, temperature can have decreases or increases in 0.5, 1, 2, 4, 8, or 10° Celsius increments.

In some embodiments of the present disclosure, the target-specific primers comprise a nucleotide modification in the 3′-end or 5′-end or across the sequence. In some embodiments, the length of target-specific portion of the primer can be about 15 to 40 bases. In some embodiments, the Tₘ of each target-specific primer can be about 55° C. to about 72° C.

In some embodiments, the disclosure features a target enrichment and multiplex barcoding amplification approach for target-specific nucleic acid amplification of microbial and viral pathogens using indexed target-specific primers. In some embodiments, the disclosure features a target enrichment and multiplex barcoding amplification approach for target-specific nucleic acid amplification of genomic material related to cancer/genetic disorders, drug resistance, forensics, allergens and other biological applications, In some embodiments, the selected indexed target-specific primers contact and hybridize to target nucleic acid sequences that can be related to disease. In one embodiment, indexed target-specific primers hybridize to nucleic acid sequences in the test reaction, which have different sizes. In some embodiments, amplicon size selection can be used to sequence amplified products of a certain length range. In some embodiments, amplicons of 100 to 250 base pairs range in length can be sequences. In some embodiments, amplicons of 150 to 300 base pairs, or amplicons of 120 to 350 base pairs, or amplicons of 200 to 500 base pairs range or greater length range can be sequenced.

In some embodiments, any of the method steps can be removed or can be repeated. In some embodiments, purification steps can be added for generating optimal results. These procedures are non-limiting and a skilled person of the art can readily add, remove or repeat the steps for optimal results.

The ability to increase the number of indexed target-specific primers in a multiplex PCR allows simultaneous amplification of a large number of nucleic acid targets while decreasing the amount of input DNA, labor and time. This is especially advantageous when the amount of starting input nucleic acid material is limited.

In some embodiments of the disclosed method, the primer design methodology selects the candidate target-specific primers based on this stepwise procedure: (1) extraction of genomic sequence around each targeted variant position; (2) for each variant in the target sequence, design target-specific forward strand and reverse strand target-specific primers with proper GC content, Tₘ, and varying distances from each targeted variant; (3) for each primer, searching target genome sequences for off-target matches; filter primers and keep those primers that pass the off-target threshold; (4) search the 3′-end portion of each primer for complementary matches with primer sequences of the set; filter primers progressively where the primer with its 3′-end having most complementary matches is removed first; (5) synthesize primers and run the entire wet-lab experiment comprising next-generation sequencing; calibrate the performance of each primer and filter out primers of undesired performance. In some embodiments, the primer selection procedure steps 2 to 4 and steps 2 to 5 are repeated until each target variant is covered by at least one forward strand target-specific primer and one reverse strand target-specific primer in the primer set.

In some embodiments, the disclosure features a primer design methodology that eliminates low compatibility primers that form artifacts such as primer-dimers in a highly multiplexed PCR that inhibit efficient amplification. Such elimination system removes or significantly minimizes the non-productive artifacts such as primer-dimers. Removal of low-compatibility and problematic primers significantly improves the overall performance and efficiency of highly multiplex PCRs in addition to downstream processes such as high throughput sequencing. Artifacts and primer dimers cause significant failure in obtaining optimal sequence results and a significant portion of the sequencing reads can be non-specific and non-informative.

In some embodiments, the primer selection methodology features a primer compatibility score both in regard to primer-primer interactions and specific target nucleic acid hybridization without non-specific priming or hybridizing to off-target regions. A higher compatibility score for a candidate target-specific primer characterizes specific hybridization to target nucleic acid with no or minimal interaction with other primers in the primer set. Primers that do not meet the compatibility score that is to say are above the minimum threshold are removed. In various embodiments of the disclosed method, a compatibility score is calculated for at least 80, 90, 95, 98, 99, or 99.5% of the possible combinations of candidate primers in the set. The compatibility score in primer selection is calculated based on a number of parameters such as target amplicon GC content, target amplicon melting temperature, target amplicon heterozygosity rate, complementary rate of the candidate primer for the target region, candidate primer size, target amplicon size and amplification efficiency. Due to the fact that several aspects are involved in determining the compatibility score, an average score is calculated based on multiple parameters and average can be variable for particular applications. The primer selection methodology will keep eliminating the low-compatibility primers, and the elimination process is repeated to equal or below minimum threshold till an optimal selection primer group is achieved that generates a highly multiplex target amplification PCR with no or minimized primer-dimers.

In some embodiments, the primer selection methodology features a primer compatibility score both in regard to primer-primer interactions and specific target nucleic acid hybridization without hybridizing to off-target regions. The primers that have low compatibility score that is to say above the minimum threshold will be eliminated. However, if there are limitations in primer selection in certain applications, the minimum threshold can be increased to a higher level of second threshold to facilitate primer selection for the primer group. In some embodiments the selection process is repeated until candidate primers are selected that are equal or under the second level of minimum threshold.

In an embodiment, the disclosed method features a multiplex amplification and target enrichment by utilizing indexed target-specific primers (in combination with indexed universal primers) that contact target nucleic acid sequences of genomic material of samples related to cancer/genetic disorders, drug resistance, forensics, allergens, microbial, fungal, parasitic, viral and other biological applications, wherein primer dimers can be reduced or minimized by adjusting different parameters such as duration of annealing steps, increase or decrease of temperature increments combined with number of cycles. In some embodiments, the primer concentrations can be lowered, and annealing temperature and duration can be increased to allow specific amplification (the primers have more time interval to hybridize to target nucleic acids) in addition to reduced or minimal primer-dimers. In some embodiments, the concentration of primers can be 500 nM, 250 nM, 100 nM, 80 nM, 70 nM, 50 nM, 30 nM, 10 nM, 2 nM, 1 nM or lower than 1 nM. In some embodiments, the annealing temperature can be 1 minute, 3 minutes, 5 minutes, 8 minutes, 10 minutes or longer. In some embodiments, the amplification with longer annealing time uses 1 cycle, 2 cycles, 3 cycles, 5 cycles, 8 cycles, 10 cycles or more followed by standard annealing durations.

In one aspect, the disclosed method comprises the step of amplifying selective target nucleic acid sequences of samples related to cancer/genetic disorders, drug resistance, forensics, allergens, microbial, fungal, parasitic, viral and other biological applications. In some embodiments, the method comprises the step of contacting the nucleic acid sample with indexed target-specific primers in presence of indexed barcoded universal primers in a test reaction. In some embodiments, the method comprises the step of determining the presence or absence of target amplification product. In some embodiments, the method comprises the step of determining the sequence of the amplified target products. In some embodiments, the method identifies the microorganism to strain or sub-strain level. In some embodiments, less than 50, 40, 30, 20, 10, 5, 0.5, or 0.1% of the amplified products are primer-dimers or artifacts. In one embodiment, there can be more than one set of target-specific primers as an example there can be two sets of target-specific primers for two test reactions, 3 sets for 3 test reactions or 5 sets for 5 test reactions or more. In some embodiments for practical reasons such as limitations in primer design or selection, the sample may also be split into multiple parallel multiplex test reactions with multiple sets of target-specific primers.

In various embodiments, concentration of each primer can be 500 nM, 250 nM, 100 nM, 80 nM, 70 nM, 50 nM, 30 nM, 10 nM, 2 nM, 1 nM or lower than 1 nM. In various embodiments, primer concentration of each primer can be between 1 μM and 1 nM, between 1 nM and 80 nM, between 1 nM and 100 nM, between 10 nM and 50 nM or 1 nM and 60 nM. In some embodiments, the GC content of target-specific primers can be between 40% and 70%, or between 30% and 60% or 50% and 80% or 30 and 80%. In some embodiments, primer GC content range can be less 20%, 15%, 10% or 5%. In some embodiments, the melting temperature (Tₘ) of the target-specific primers can be between 55° C. and 65° C., or 40° C. and 72° C., or 50° C. and 68° C. In some embodiments, the melting temperature range of the primers can be less 20° C., 15° C., 10° C., 5° C., 2° C. or 1° C. In some embodiments, the length of the target-specific primers can be between 20 and 90 bases, 40 and 70 bases, 20 and 40 bases or 25 and 50 bases. In some embodiments, the range of length of the primers can be 60, 50, 40, 30, 20, 10, or 5 bases. In some embodiments, the 5′-region of the target-specific primer is a universal priming site that are not complementary or specific for any target nucleic acid regions.

In one aspect, the present disclosure is directed to a kit that comprises indexed target-specific primers in a group; the primers are designed and selected based on criteria described to have minimal primer-primer interactions or non-specific priming. In another embodiment, the kit can be formulated for detection, screening, diagnosis, prognosis and treatment of disease. In another embodiment, the kit can be formulated for detection of drug resistance. In some embodiments, the kit can be used for bacterial, fungal, parasite and viral screening, detection, identification, genotyping, subtyping, and surveillance. In some embodiments, the kit can be used for analysis of samples related to cancer/genetic disorders, forensics, drug resistance, pharmacogenetics and other biological applications. In some embodiments, the kit can be used for detection of allergens.

In some embodiments, the disclosed method comprises the steps of: (1) contacting a set of indexed target-specific primers with target nucleic acid sequences in the presence of indexed barcoded universal primers and hybridizing to target nucleic acid sequences in each sample in the test reaction; (2) subjecting the test reaction to amplification under optimal amplification conditions generate amplicons containing quadruple combinatorial indexes; (3) pooling together the amplified products from each individual sample; (4) subjecting a portion of the pooled amplified products to bead cleanup to remove possible primer-dimers to create enriched amplified products; (5) subjecting a portion of the enriched amplified products to standard normalization and quantification; and (6) sequencing the amplicon by next-generation sequencing. The method may further comprise additional steps, such as purification. In one aspect, highly multiplex PCR is utilized for the method disclosed. In some embodiments, between 1 and 10 cycles of PCR can be performed for PCR; in some embodiments between 1 and 15 cycles or between 1 and 20 cycles or between 1 and 25 cycles or between 1 and 30 cycles, between 1 and 35 cycles or more can be performed.

In another embodiment, the disclosed method can be used in a multiplex fashion when amplifying more than two targets and is not limited to any number of multiplexing.

In some embodiments, the amplification product can be sequenced by next-generation sequencing platforms. Next-generation sequencing is referred to non-sanger based massively parallel DNA nucleic acid sequencing technologies that can sequence millions to billions of DNA strands in parallel. Examples of current state of state-of-art next-generation sequencing technologies and platforms are Illumina® platforms (reversible dye-terminator sequencing), 454@ pyrosequencing, Ion Semiconductor sequencing (Ion Torrent), PacBio® SMRT sequencing, Qiagen® GeneReader sequencing technology, Element Biosciences® Sequencing platforms, and Oxford Nanopore® sequencing. The present disclosure is not limited to these next-generation sequencing technologies examples.

### EXAMPLE 1

### Evaluation of Combinatorial Quadruple Indexing Method to Remove Reads Due to Index Hopping on Three Synthesized DNA Targets

### Materials and Methods

Samples and Assay Design: Three target DNA templates were synthesized (IDTDNA, Coralville, IA). For amplification and indexing, the following primers were synthesized: three indexed forward target-specific primers with three different indexes, three indexed reverse target-specific primers with three different indexes, three P5 primer (indexed universal primer) with three different indexes, and three P7 primer (indexed universal primer) with three different indexes.

Amplification: One-step multiplex barcoding PCR was performed on the synthetic DNA templates by indexed target-specific primers in the presence of P5 and P7 primers (indexed universal primers), DNA polymerase, dNTP and PCR buffer.

Next-generation sequencing: All the amplicons were pooled into one tube and purified by SPRIselect beads (Beckman Coulter, Brea, CA). The purified sample concentration was measured on a Qubit™ 3 and the concentration was normalized for sequencing. The library was sequenced with Illumina® MiniSeq™ system using an Illumina® Mid Output sequencing kit.

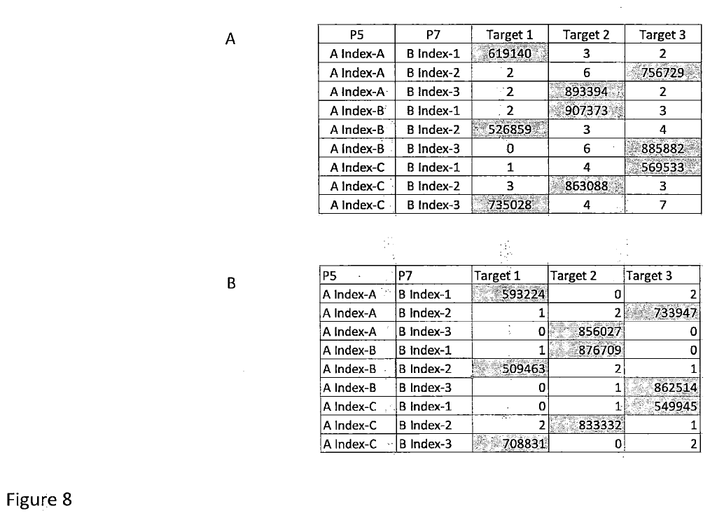

Results: FIG. 8A lists the reads count for each target with different A/B index-pairs. The numbers for permissible combinations are highlighted with grey background. There are 57 unintended reads. After removing reads with impermissible combinations of indexes, there are 16 unintended reads left (FIG. 8B). The correction process removes 72% unintended reads. The few number of unintended reads might be due to synthetic manufacturing errors, which may be resolved by more diverse sequences in the indexes. This result demonstrates that the combinatorial quadruple indexing approach mitigates index hopping significantly, and can be reduced further with more diverse indexing sequences. The numbers with grey background in FIG. 8A are lower than their corresponding wells in FIG. 8B, because reads matching perfectly with target index were passed and reads with same A/B index-pair but non-matching target indexes were removed.

### EXAMPLE 2

### Index Hopping Assessment on Sexually Transmitted Infected Clinical Samples by Multiplex Barcoding Amplification and Combinatorial Quadruple Indexing method

### Materials and Methods

Samples: 480 DNA samples that had already been tested for human papillomaviruses (HPV) were used for the disclosed method.

Assay Description: The HPV-STI assay applied for this experiment utilizes a combination of type-specific primers targeting 29 HPVs (including HPV.68a and 68b) and 13 STIs, including Chlamydia trachomatis serovars and the GAPDH internal control. Amplification and barcoding/indexing of each sample was simultaneously performed in a single tube and a single. PCR reaction. The high-risk HPV types include HPV 16, 18, 31, 33, 35, 39, 45, 51, 52, 56, 58, 59, 66, 68a, 68b, 73, 26, 53, 82, and the low-risk HPV types include HPV6, 11, 40, 42, 43, 44, 55, 61, 81, 83. The 13 STIs include Chlamydia trachomatis, Treponema pallidum, Mycoplasma genitalium, Trichomonas vaginalis, Neisseria gonorrhoeae, HSV-1, HSV-2, Mycoplasma hominis, Ureaplasma urealyticum, Ureaplasma parvum, Varicella zoster virus, Haemophilus ducreyi.The Chlamyida serovars include L1, L2, B, D, E, F and G.

Multiplex Barcoding Amplification: One-step multiplex barcoding PCR by combinatorial quadruple indexing was performed on 480 clinical samples (5×96 PCR plates) with 29 HPV, 13 STI and an internal control indexed target-specific primers in the presence of indexed universal primers, sample DNA, DNA polymerase, dNTP and PCR buffer. Each generated amplicon contained quadruple indexes.

Next-generation sequencing: After amplification, the clinical samples for each plate were pooled into one tube. A portion of the samples was then purified with SPRIbeads (Beckman Coulter, CA, USA) according to the manufacturer's instructions. The purified sample concentration was measured with Qubit™ 3 and the concentration was normalized for sequencing. The library was sequenced with Illumina® MiSeq™ system using an Illumina® Mid Output sequencing kit. One library was sequenced twice with two different concentrations. A total of five sequencing runs were performed for 5 libraries, wherein each library consisted of 96 samples.

### Results

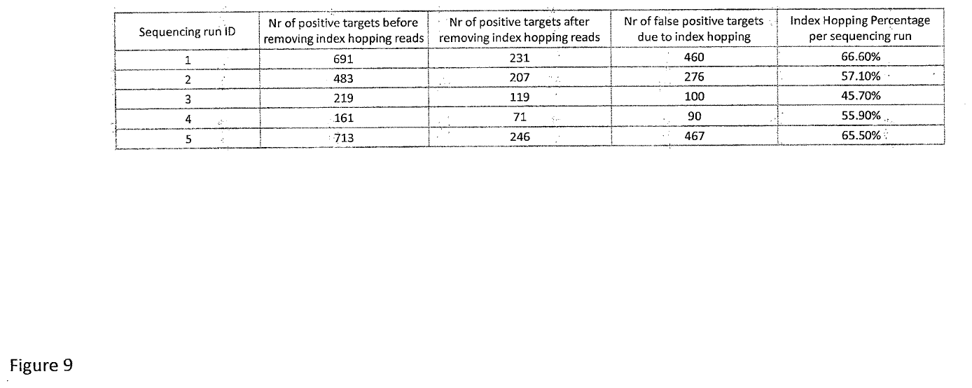

FIG. 9 lists the 5 libraries where column 2 lists the number of positive (read count>0) targets before removing index hopping reads, column 3 lists positive (read count>0) targets after index hopping removal, column 4 lists the percentage of false positive due to index hopping for each library. Libraries 1-5 generated 460(66.6 %), 276 (57.1%), 100 (45.7 %), 90 (55.9 %) and 467 (65.5 %) false positive targets due to index hopping, which were removed as impermissible combinations of indexes by the data analysis software. The reads matching perfectly with the quadruple target indexes are assigned to each designated sample and reads with the same A/B index-pair but non-matching target indexes were eliminated.

The results from the five libraries indicate that the combinatorial quadruple indexing approach mitigates false positives due to index hopping significantly, where many samples that would have been reported as positive are indeed negative.

Now, therefore, the following is claimed:

1. A method of indexing at least one nucleic acid sample, comprising the steps of:
for each sample, hybridizing a plurality of indexed target-specific primers with the sample in the presence of indexed universal primers to form a plurality of test reactions in a single reaction container, wherein at least one target-specific primer is configured to bind to at least one target nucleic acid sequence and wherein nucleic acid targets are first labeled with indexed target-specific primers in primer extensions of amplification;
simultaneously amplifying the index-labeled products using indexed universal primers, wherein each test reaction is subject to amplification conditions configured to generate amplicons containing four combinatorial indexes, wherein one index (A) is located at the 5′ end of amplicons at Watson (forward) strand, one index (B) is located at the 5′ end of amplicons at Crick (reverse) strand, one index (X) is located adjacent to target-specific primers at the Watson (forward) strand, and one index (Y) is located adjacent to target-specific primers at the Crick (reverse) strand; and
subjecting at least a portion of the pooled amplicons to next-generation sequencing.

2. The method of claim 1 wherein, within a set of samples, the same X index (next to target-specific primers at the Watson strand) is shared within more than one sample in the same set, the same Y (next to target-specific primers at the Crick strand) index is shared with more than one samples in the same set, but wherein the combination of X and Y indexes is unique for each sample within the same set, and wherein the same A index (at 5′ end of amplicons at the Watson strand) is shared within more than one sample in the same set, and the same B index (at 5′ end of amplicons at the Crick strand) is shared with more than one sample in the same set, but wherein the combination of A and B indexes is unique for each sample within the same set.

3. The method of claim 1, wherein each indexed universal primer comprises:
a universal priming portion at the 3′-end;
an index portion in the middle; and
a universal priming portion at the 5′-end.

4. The method of claim 1, wherein each indexed target-specific primer comprises a universal priming portion, an index portion and a specific sequence portion directed to a target nucleic acid sequence.

5. The method of claim 1, wherein the generated amplicons have four indexes.

6. The method of claim 1, wherein the amplification is performed in two-steps comprising a first step wherein X indexed primers and Y indexed primers are used to amplify targets to form first amplicons, and using A indexed primer and B index primer to amplify the first amplicons and add A and B indexes to create second amplicons with four indexes.

7. The method of claim 1, wherein each nucleic acid sample is obtained from a subject, human, plant, food, one or more plants, or an environmental source.

8. The method of claim 1, wherein the indexed target-specific primers comprise at least two primers, wherein each primer is configured to amplify a distinct nucleic acid target.

9. The method of claim 1, further comprising the step of pooling the enriched amplicons from each sample immediately prior to bead cleanup.

10. The method of claim 1, further comprising the step of quantifying each nucleic acid target in each sample after sequencing the enriched amplicons.

11. The method of claim 1, wherein each index comprises a distinct sequence.

12. A method of indexing at least one nucleic acid sample, comprising the steps of:
for each sample, hybridizing a plurality of indexed target-specific primers with the sample in the presence of indexed universal primers to form a plurality of test reactions in a single reaction container, wherein at least one target-specific primer is configured to bind to at least one target nucleic acid sequence, and wherein nucleic acid targets are first labeled with indexed target-specific primers in primer extensions of amplification; and
simultaneously amplifying the index-labeled products using indexed universal primers, wherein each test reaction is subject to amplification conditions configured to generate amplicons containing four combinatorial indexes, wherein one index (A) at 5′ end of amplicons at Watson (forward) strand, one index (B) at 5′ end of amplicons at Crick (reverse) strand, one index (X) next to target-specific primers at the Watson (forward) strand, and one index (Y) next to next to target-specific primers at the Crick (reverse) strand;
pooling the resulting amplicons; and
subjecting at least a portion of the pooled amplicons to next-generation sequencing.

13. A kit, comprising multiplex indexed target-specific primers configured to bind to nucleic acid sequences specific to a target selected from the group consisting of: cancer, genetic disorders, allergens, forensic testing targets, microbial and viral species, and any nucleic acid-based biological target.
